(19) United States
(12) Patent Application Publication
(10) Pub. No.: US 2016/0022147 A1
(43) Pub. Date: Jan. 28, 2016

(54) METHOD AND DEVICE FOR MONITORING VITAL FUNCTIONS

(71) Applicant: SENSPEC GMBH, Rostock (DE)

(72) Inventor: Axel KULCKE, Am Salzhaff (DE)

(73) Assignee: SENSPEC GMBH, Rostock (DE)

(21) Appl. No.: 14/774,901

(22) PCT Filed: Mar. 4, 2014
(86) PCT No.: PCT/EP2014/054118

§ 371 Date: Sep. 11, 2015

(30) Foreign Application Priority Data
Mar. 11, 2013 (EP) 13158577.0

(51) Int. Cl. A61B 5/00; A61B 5/024; A61B 5/1455

(52) U.S. Cl. CPC A61B 5/0075; A61B 5/14551; A61B 5/02438; A61B 5/6826; A61B 5/6816

(57) ABSTRACT

A method for monitoring vital parameters of a living being, wherein broad-band light is beamed into the tissue of a living being, a spectrum of the beamed-in light is recorded, and absorption is evaluated. At least one first parameter is determined on the basis of the absorption values of a first spectral range, and at least one second parameter is determined on the basis of the absorption values of a second spectral range. The first parameter is compared to the second parameter, and at least one vital parameter is determined with the aid of this comparison.

The present invention relates to a method and a device for monitoring vital functions of patients.

Various methods and devices for recording and/or monitoring the vital functions of patients are known from the state of the art. Non-invasive devices and methods are preferably used in particular in cases of emergency, during first aid, during operations and also for long-term monitoring or sleep monitoring. Devices and methods of this type should deliver fast, accurate results in order to be informed as quickly as possible about the state of health of the patient or to make decisions about further measures.

Pulse oximeters which measure the oxygen saturation of blood are, for example, known from the prior art. Typically, light is emitted into a tissue, and the absorption is measured. An earlobe or a finger is, for example, placed in a holder of a device of this type. The measurements are made both in the red and in the near-infrared range of light, typically one measurement being taken at a wavelength of 660 nm and one at 940 nm.

Pulse spectrometers that are appropriate for evaluating the pulse on the basis of its amplitude by means of spectroscopy, as e.g. disclosed in WO 2011/161102 A1, are also known.

A widespread problem with methods and devices of this type is that the measurements are imprecise or that the measurements or the results are corrupted. Since the measurements are made at individual wavelength ranges, they are extremely sensitive to changes in, for example, the environmental conditions of the measured object. An amplitude-based evaluation of this type is, moreover, subject to artifacts of movement. If a patient moves, the blood can, for example, be accelerated or slowed, or a sensor attached to the body can slip. There is, furthermore, a risk of external influences, in particular from electrical devices, magnetic or electric fields, or voltage variations within the devices. The worse a patient's vital functions are, the more difficult it is to obtain accurate results, since background noises, known as artifacts, can become very large in comparison.

Different approaches to excluding movement artifacts of this kind from the measurement signal exist. US 2011/0245654 for example discloses a method which attempts to filter out the movement artifacts with an adaptive filter. Near infrared light (NIR) and red light are applied, and the optical density is measured. The measured values are compared with comparison values in a numerical method, and the oxygen saturation of the blood determined by means of the maximum excursion. This method is expensive and complex, and requires high computing power.

It is therefore an object of the invention to overcome the disadvantages of the prior art. In particular, a method and a device are to be provided that minimize complicated computing processes, are less subject to movement artifacts, and thus avoid false alarms.

This object is achieved through the method and devices defined in the independent patent claims. Further embodiments emerge from the dependent patent claims.

The method according to the invention is provided for monitoring vital parameters of a living being. A vital parameter is, in particular, the pulse rate or the oxygen saturation of the blood. It is, however, also conceivable for the method to be employed for monitoring further parameters such as blood sugar or other chemical components of the blood or of other liquids. The method comprises the following steps. Broadband light, preferably pulsed light, is beamed into tissues of a living being. The light here preferably comprises wavelengths in a spectral range from 400 nm to 850 nm. A spectrum of the light returned from the tissue is recorded. The light can here either be reflected or transmitted in the tissue. An evaluation of the recorded spectrum is then performed. At least a first parameter is determined on the basis of the first spectral range within the spectrum to evaluate the absorption. Additionally, at least a second parameter is determined on the basis of at least a second spectral range. The second spectral range is larger than the first spectral range, and at least partially contains the first spectral range. The first parameter is compared with the second parameter, and at least one vital parameter determined on the basis of the comparison.

Both oxygenated hemoglobin (HbO2) and reduced hemoglobin (HHb) comprise several isosbestic points in this wavelength range. Points of this type are particularly found in the wavelength range from 500 nm to 600 nm. In this range the absorption coefficients of the hemoglobin are higher, and the ratio between absorption of the hemoglobin and scattering of the light in the tissue is increased. As a result of the pulse, both the amount of arterial blood in the tissue is increased, or changed continuously, as is the oxygen saturation in the tissue. A basic level of absorption from the elements of the tissue such as skin and bone, which do not change or only do so slowly, nevertheless remains.

The spectra typically di play the absorption of the light beamed into the blood and tissue. The parameter that is determined from the first, smaller, spectral range undergoes marked variations depending on the pulse or on the oxygen saturation in the blood, in particular when the range includes those wavelengths with increased absorption of the oxygenated hemoglobin HbO2 between two isosbestic points. The second parameter, which is obtained from a broader spectral range, provides a basis that is only subject to small and/or slow changes. Through a comparison of these two parameters, a value can be determined that permits, for example, a conclusion to be drawn about the saturation of the blood with oxygen. If the evaluation is performed over a broad spectral range with a plurality of wavelengths, the effect of the respective changes of individual components is smaller. The method according to the invention thus does not measure on the basis of pure amplitude. Rather it is an amplitude-based measurement in a partial range of the spectrum normalized by a measurement over a broader range. The partial range here includes wavelengths in which the absorption changes in a constant manner depending on the oxygen saturation, typically increasing with higher saturation. The second range includes segments in which the absorption increases with higher oxygen saturation and segments in which the absorption falls with higher saturation. The parameter measured in the second range therefore reflects the scattering in the tissue or the absorption in the non-oxygenated blood more strongly than the measurement in the first range. The measurement over the larger range therefore depends on the oxygen saturation to a much lower extent than the measurement over the first, narrower range. By comparing these values, an oxygen-based measurement can be achieved rather than an amplitude-based one.

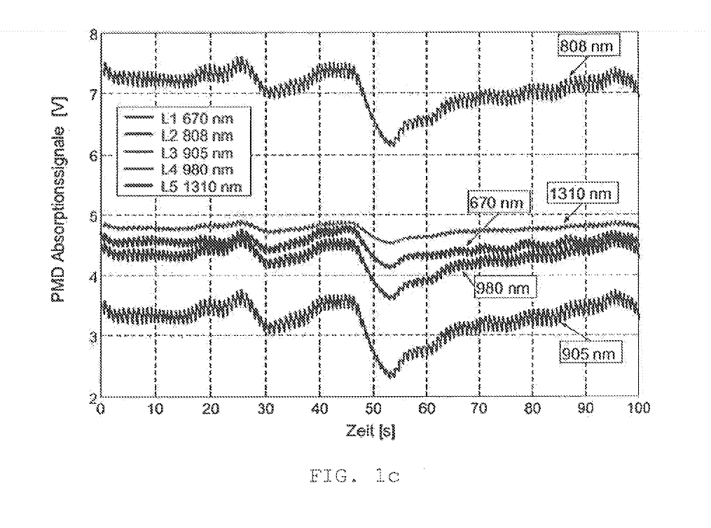

Preferably the first range is selected such that the absorption increases with a higher oxygen saturation, which means that the HbO2 absorption coefficient is greater than the HHb absorption coefficient. In a preferred embodiment, the first spectral range substantially corresponds to a range between at least two neighboring isosbestic points, over which the relationship of the absorption to the oxygen saturation remains the same, for example increasing. Isosbestic points are located for example at 505 nm, 522 nm, 548 nm, 570 nm and 586 nm (see FIG. 2 in this connection).

The first spectral range can be composed of at least two partial ranges. Preferably each of these partial ranges comprises absorption coefficients whose relationship to the oxygen saturation remains the same, for example increasing. If the partial ranges are chosen suitably, the absorption adds. This allows a conclusion to be reached even when the oxygen saturation is low.

One particularly advantageous possibility for comparing the first parameter with the second parameter is given by the formation of the quotient of the first and the second parameters. A quotient of this sort can be scaled as desired, and represents a dimensionless value. Through the formation of a quotient, a basic absorption, caused for example by hemoglobin absorption, absorption in the skin, the tissue or by bones, can be filtered or excluded from the measurement.

The second spectral range can comprise a range of wavelengths at least twice as large as the first spectral range. Through a broad measurement of this sort of the second spectral range, a value is obtained which is only subject to small variations depending on the oxygen saturation, or which only changes slowly.

Preferably the second spectral range comprises a range of wavelengths that comprises a plurality of isosbestic points. This favors a value that is subject to smaller variations.

Both the first and the second parameter can be determined through an integral of the absorption curve over the first or second range. Through the integration, individual values are obtained that can be combined together in an easy manner.

Preferably an oxygen signal that represents the time-resolved evaluation is generated with the method according to the invention. A representation of this sort can be recorded with a frequency of between 50 and 150 Hz. This allows the oxygen saturation to be represented against time. It is possible for example in this way to detect a hypoxia. Oxygen values in the tissue fall, and the comparative values undergo a significant change since, for example, the base absorption varies from a longer-term value.

It is also possible to determine the pulse rate from the oxygen signal, since the oxygen saturation is directly affected by the pulse, that is by the freshly supplied arterial, i.e. oxygen-enhanced blood. Conclusions can thus be drawn about the oxygen saturation and also about the pulse. A combination of the evaluation with a display appliance, or further processing of the values in relation to one another, is now possible.

The pulse component in the absorption constitutes only a small part of the total optical signal—about 1% to 5% in a healthy human. In critical patients, this component can fall by a factor of 10 or more. A pulse or an oxygen saturation that is to be classified as critical can nevertheless be detected by the method according to the invention, or a pulse can be calculated from the change in the oxygen saturation.

This is possible according to the present method, since it is not the absolute signal that is evaluated, but the ratio of the different absorption of the individual waves in the respective spectral ranges through the formation of a quotient.

The result of the comparison between the first parameter and the second parameter, which can for example be present as a vital parameter, can be compared with a purely amplitude-based signal. It is conceivable that a signal of this type is determined by means of the absorption of light that is beamed in, wherein the absorption is for example measured at a specific wavelength. Wavelengths common in the prior art are, for example, 660 nm or 940 nm for the measurement of the oxygen saturation of the blood; other wavelengths are conceivable for other properties of the blood, and are known to the expert in the field. Typically a wavelength or a range of the light beamed in comprising several wavelengths, for example the range of wavelengths between two isosbestic points, is selected for generating an amplitude-based signal. A signal of this type can, for example, be obtained from the first or second parameter determined according to the present method. The comparison with a second, independent, amplitude-based signal, for example with the signal of a pulse oximeter, can, however, also be considered.

The solution according to the invention makes an oxygen-based signal and an amplitude-based signal available. A measure of the reliability of the signals can be determined from a comparison of these two signals, or from the degree of agreement between the two signals. It is thus for example possible to set alarms in which a change in interaction between the signals as an indication that, for example, the one signal is no longer being correctly detected, for example if the light is no longer being correctly beamed into the tissue. Time resolved recording of these two signals is also conceivable. Longer-term changes or variations can be determined in this way, or various mean values, in particular including mean values over individual intervals or periods of time, can be compared with one another.

A further aspect of the invention relates to a device for monitoring vital parameters of a living being, in particular for monitoring the pulse. Preferably the device is suitable for carrying out the method as described herein. A device of this sort comprises at least one light source for beaming light into the tissue. The light source preferably emits a broadband light spectrum.

The spectrum in particular comprises light with a wavelength in the range between 400 nm and 850 nm. The device moreover comprises a receiver for the light radiation from the tissue. The receiver also comprises means for dispersing the light and a detector, so that a light spectrum can be detected from the light returned from the tissue. The device comprises a means of evaluation. This means of evaluation is suitable for determining at least one first parameter on the basis of a first spectral range and at least one second parameter on the basis of a second spectral range within the spectrum. The second spectral range is larger than the first spectral range. The means of evaluation is also suitable for performing a comparison between the first parameter and the second parameter, and carrying out a determination of the vital parameter on the basis of the comparison. This favors the processing of the vital parameter, for example its evaluation.

Favorably the means of evaluation comprises means for integrating at least the first and the second spectral ranges. As a result at least the first and the second parameter can be determined. Values determined in this way can, for example, be processed further in a processor of the measuring device.

In a preferred embodiment, the means of evaluation comprises means for the formation of a quotient from the first and the second parameters. As a dimensionless value, a quotient is particularly advantageous for evaluation or for further processing. Through the formation of a quotient, a basic absorption can be excluded from the signal that is to be processed further. Only values that change and are therefore of interest are thus evaluated.

The means of evaluation can moreover comprise means for generating an oxygen signal, so that the evaluation can be represented with time resolution. A representation against time of this sort allows conclusions to be drawn about the progress of the vital value. In particular, the change of the value over time can be seen.

The device can comprise means of fastening, preferably means of fastening for fastening the device to a finger, the forehead, an earlobe or to a skin surface. This is particularly advantageous for cases in which measurements must be taken over a relatively long period of time.

Advantageously the light detector comprises a 2-dimensional sensor array, in particular a CMOS sensor, preferably a monolithic CMOS pixel array. A sensor of this type permits direct acquisition of the light spectrum, wherein for example each region or pixel, or each pixel array, is assigned to a different wavelength, and direct integration is permitted through adding together adjacently located sensor cells. Direct amplification and further processing of the signal is, moreover, possible.

The invention is explained in more detail below with reference to figures that merely represent exemplary embodiments. Here:

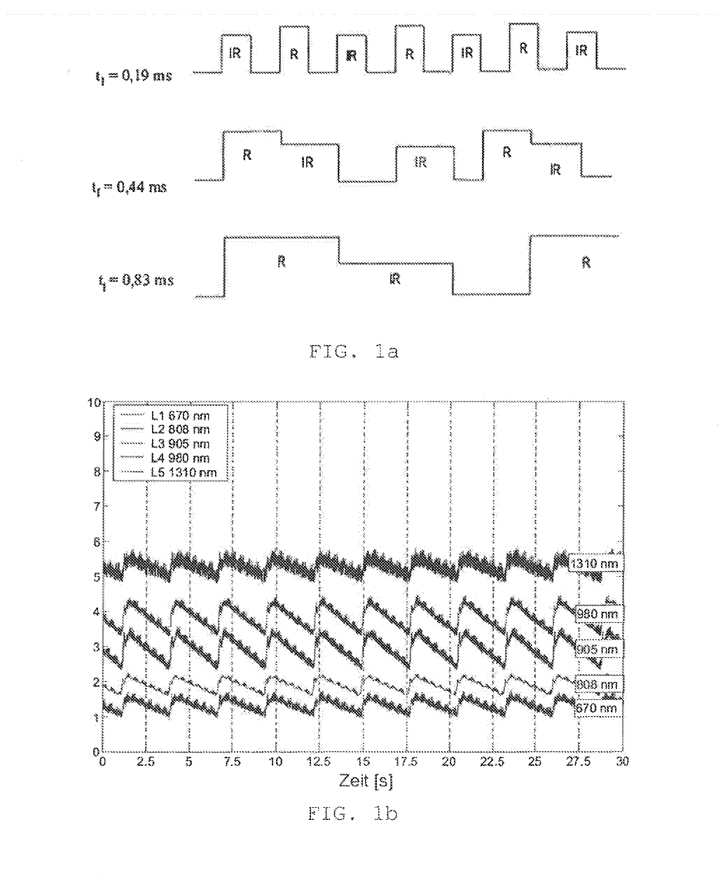

FIG. 1a: shows typical measurement curves from pulse oximeters of the prior art;

FIG. 1b: shows typical noise relationships for in vitro blood measurements;

FIG. 1c: shows typical real signals from pulse oximeters;

FIG. 2: shows the absorption curve of hemoglobin between 500 nm and 600 nm;

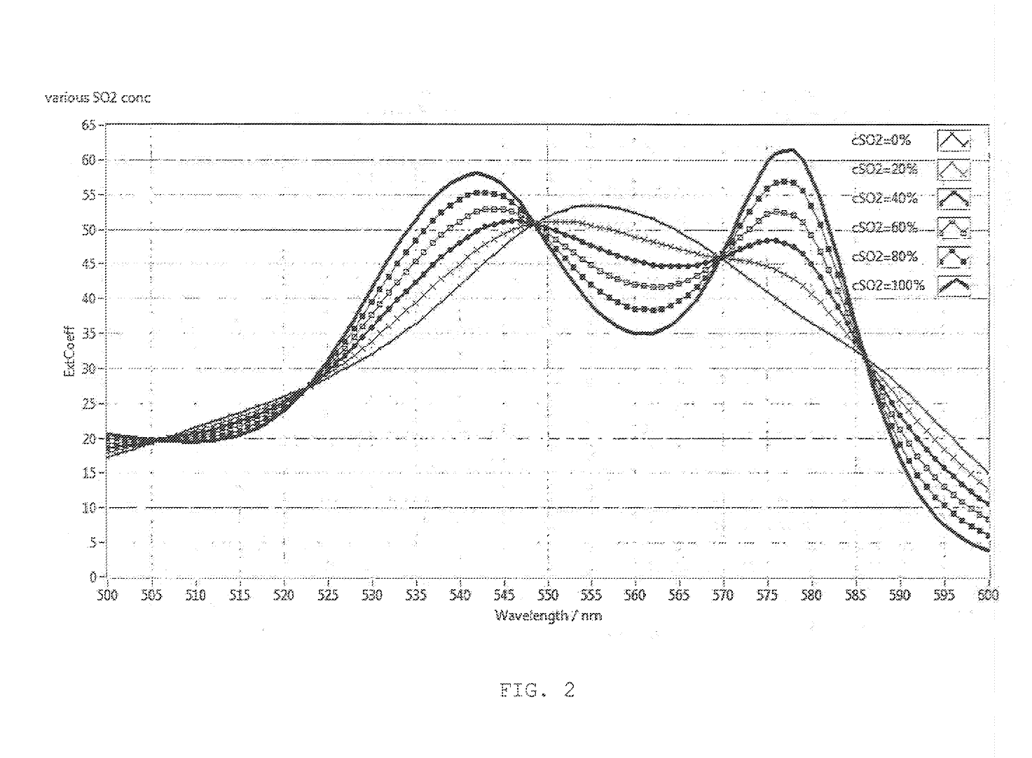

FIG. 3: shows the curve of FIG. 2, on which measurement ranges have been drawn;

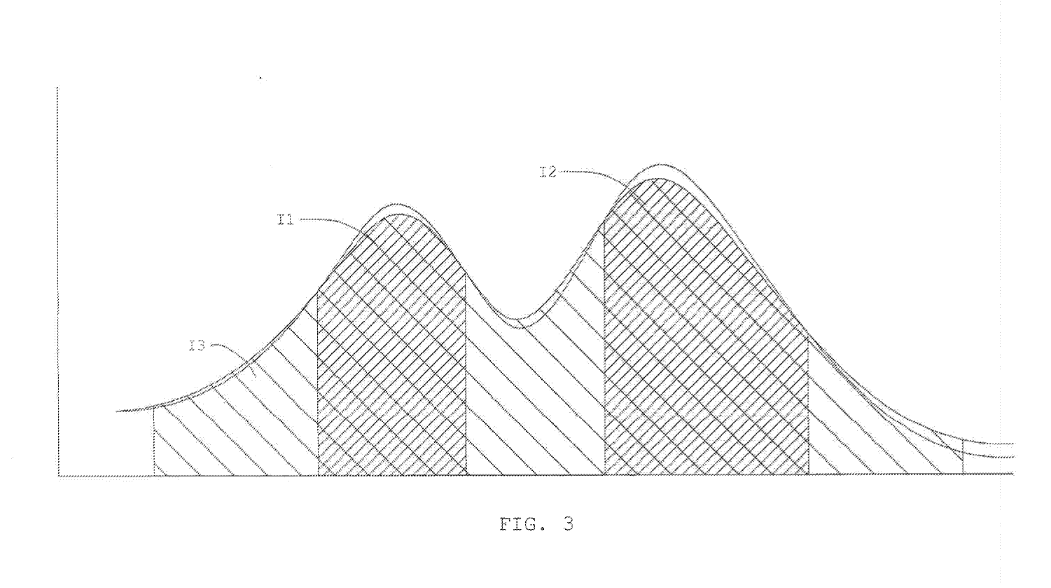

FIG. 4a: shows an undisturbed pulse curve at low frequency;

FIG. 4b: shows an undisturbed pulse curve at high frequency;

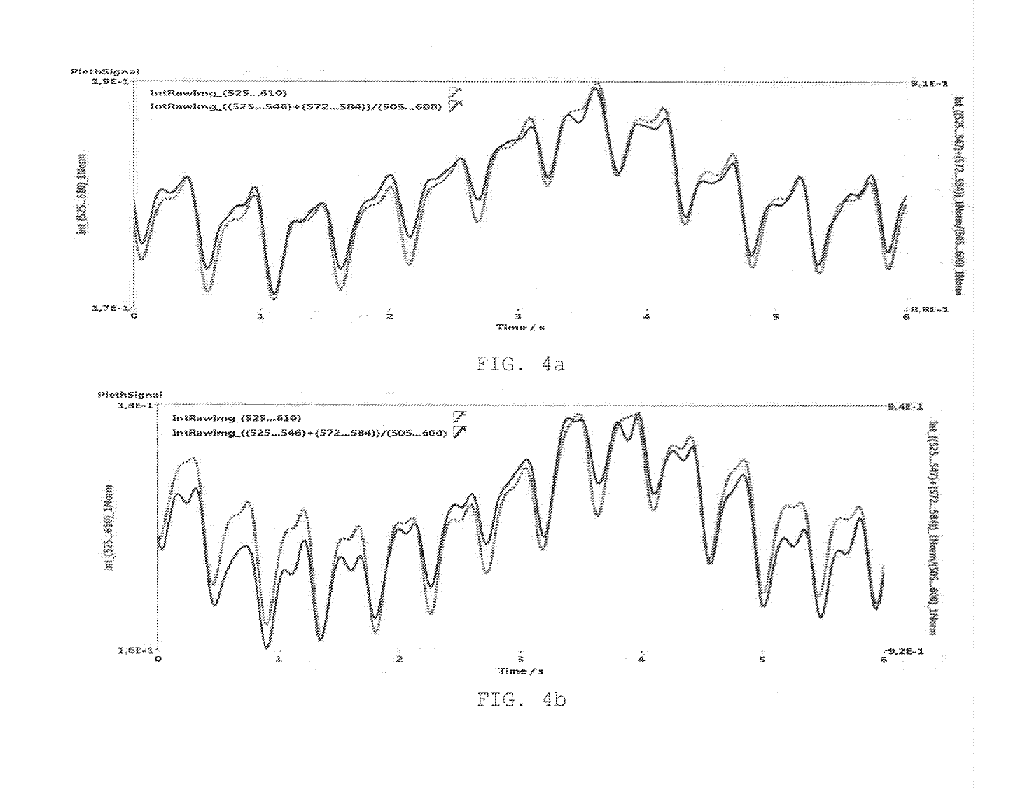

FIGS. 5a and 5b: show pulse curves with disturbances;

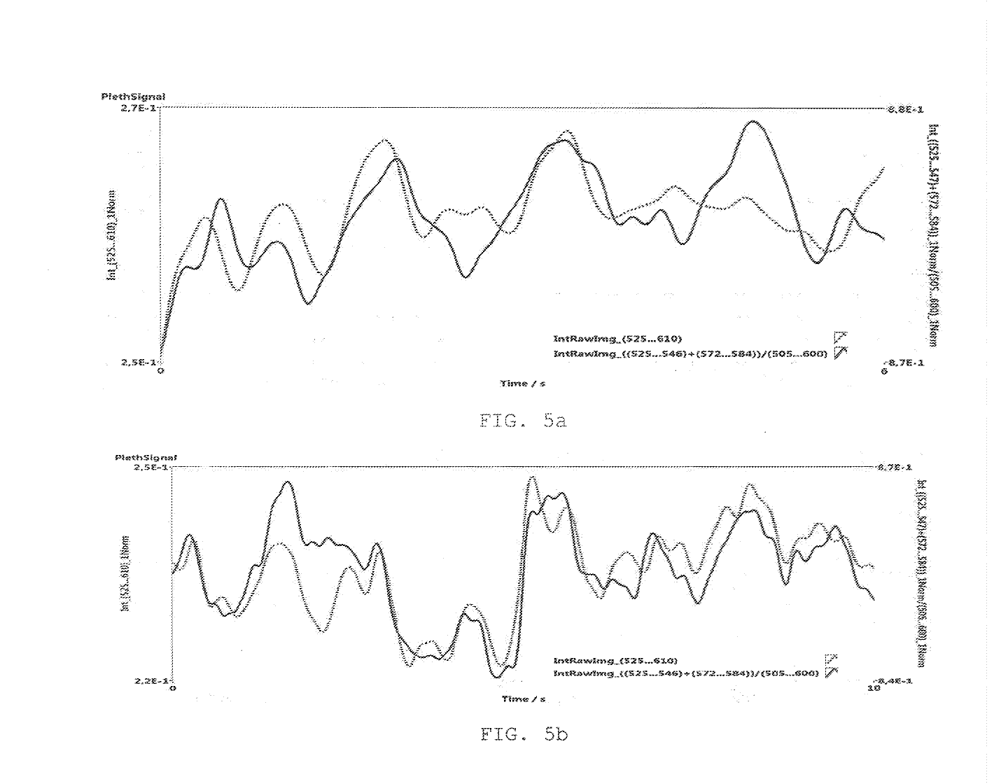

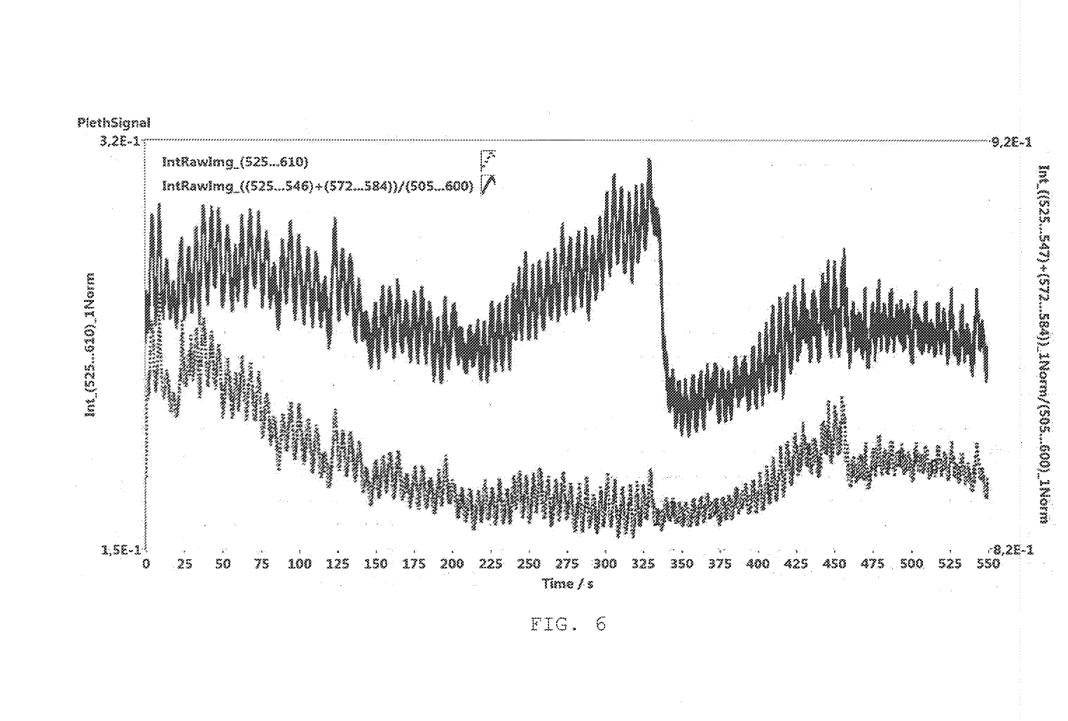

FIGS. 6 and 7: show two pleth signals over a relatively long period of time;

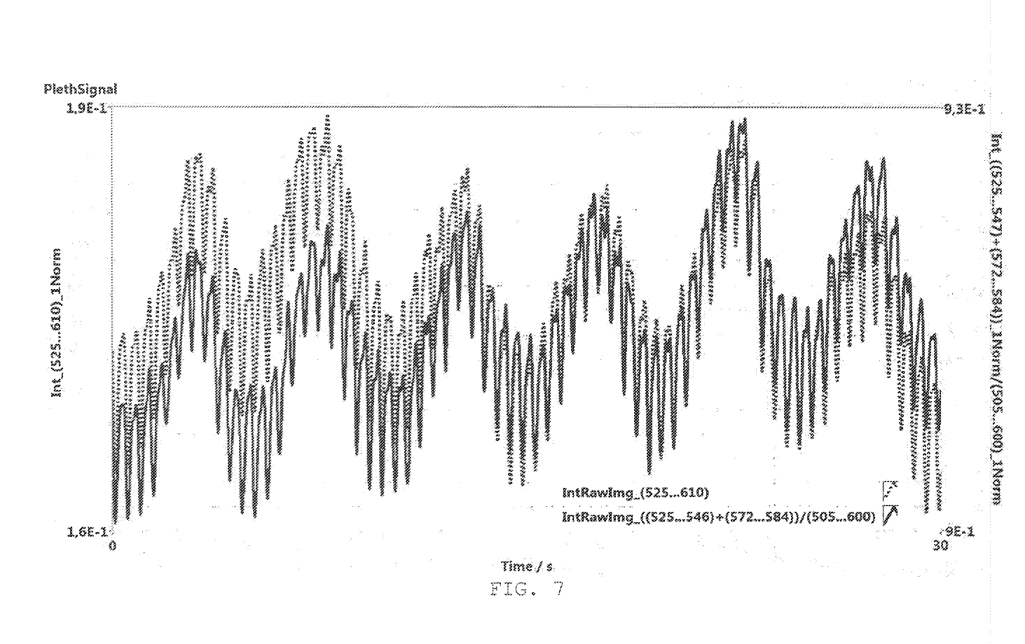

FIG. 8: shows the frequency spectrum of the pleth signal from FIG. 7;

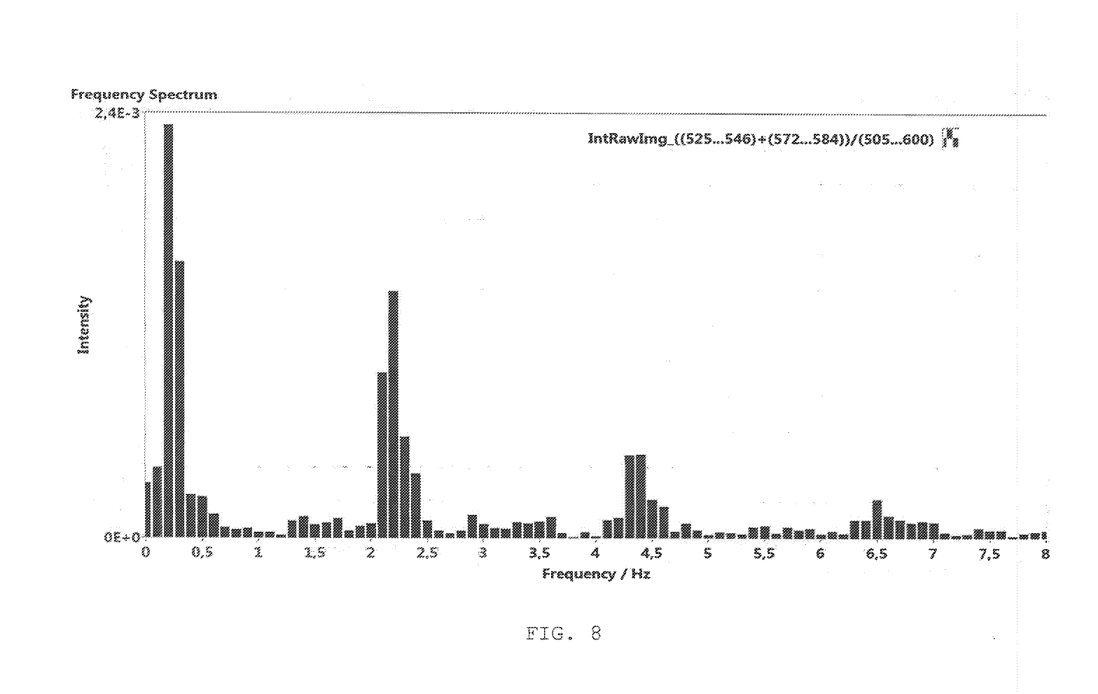

FIG. 9: illustrates a device for carrying out the method according to the invention;

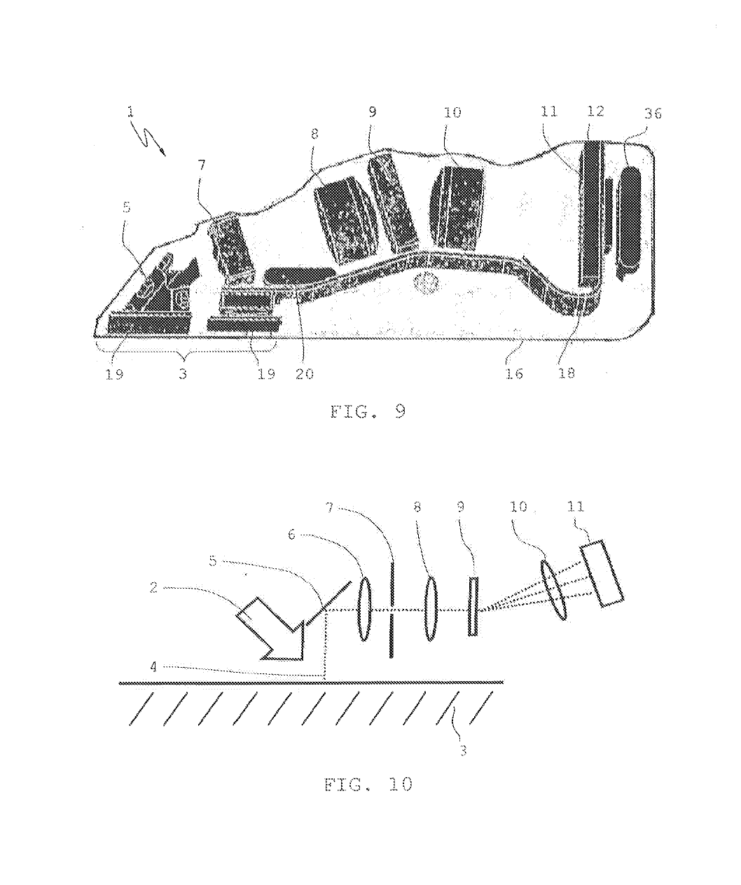

FIG. 10: shows a schematic representation of the device from FIG. 9.

FIG. 1a shows typical sequences of light recordings from various pulse oximeters available on the market. There are devices that transmit infrared and red light in alternation, with dark phases between them (first curve). Alternative measuring instruments show curves following one another (curve 3), or curves with repeating single phases (curve 2).

FIG. 1b shows typical noise relationships in technical constructions, for example a measurement at a bloodline. Each of these graphs is assigned to a particular wavelength of the light spectrum. In general, there is superimposed noise, as well as a pulsed change, caused here by a peristaltic pump.

FIG. 1c now shows the real signals for the same wavelengths from FIG. 1b, the measurements having been taken from a healthy subject. The measurement was made at the middle finger of the right arm.

FIG. 2 shows the absorption spectrum of the hemoglobin in the wavelength range between 500 nm and 600 nm for exemplary saturations of the hemoglobin with oxygen between 0% and 100%. The isosbestic points at 505 nm, 522 nm, 548 nm, 570 nm and 586 nm can clearly be seen. At an isosbestic point the relationship of increasing absorption coefficients with increasing oxygen saturation changes to falling absorption coefficients with increasing oxygen saturation, and vice versa. The absorption coefficient of, for example, the curves between the isosbestic points at 522 nm and at 548 nm, as well as the curves between the isosbestic points at 570 nm and 586 nm, is thus higher the greater the concentration of oxygen is.

FIG. 3 now shows the selected measurement ranges for the present method. The ranges marked as I1 and I2 are the ranges in which the absorption coefficient of the oxygen-saturated blood HbO2 is higher than that of the low-oxygen blood HHb. These ranges together form a first partial range of the absorption spectrum. The range marked as I3 indicates a second range that is used to determine a second parameter. The range between 505 nm and 600 nm (cf. FIG. 2 in this connection) is shown by way of example for a value of the oxygen concentration.

The curves of the partial ranges I1 and I2 are each integrated separately, at the same or different times. The coefficients are added to form a single value. The curve of the second partial region is integrated in a further step, which can be taken simultaneously or at another time. Simultaneous processing does not, however, exclude serial sampling of the individual wavelengths. A quotient can be formed from the values of these two integrals, shown by way of example in the following formula.

$$\frac{{\int }_{523}^{548} Absorbtion ( f ) d f + {\int }_{570}^{586} Absorbtion ( f ) d f}{{\int }_{505}^{600} Absorbtion ( f ) d f} = P$$

I1 and I2 are the partial regions of the first spectral range, while I3 indicates the second spectral range. P is a dimensionless coefficient that can be used for further processing.

It is of course also conceivable as an alternative that the first measurement take place in a partial range that comprises lower absorption coefficients at higher oxygen saturation of the hemoglobin. Further different parameter curves can be generated in this way. It is, for example, also conceivable that a difference is formed from the coefficients of the integration.

FIG. 4a shows by way of example the juxtaposition of dimensionless coefficients with an amplitude-based measurement of a slow and steady pulse over time. The instantaneous measurements carried out as in the method described here can be made, for example, at a frequency of between 50 and 150 Hz. The values obtained in this way allow the oxygen saturation to be plotted against time and represented in a pleth diagram. The juxtaposition of this oxygen-based evaluation (which can be recognized as the continuous line) with the amplitude-based measurement (drawn dotted on the present plot) shows significant agreement.

FIG. 4b shows measurement results corresponding to FIG. 4a for a fast pulse.

FIGS. 5a and 5b show pulse curves with disturbances resulting from movement artifacts, wherein the curves correspond to those from FIGS. 4a and 4b. The characteristic shape of the curve formed from the dimensionless coefficients is more marked when compared with the amplitude-based measurement.

FIG. 6 shows a pleth curve taken over a relatively long period of time. The clearly visible frequency is the respiration frequency. The pulse frequency can no longer be illustrated at this resolution. The amplitude-based curve (shown dotted) comprises no noticeable features. On the other hand, the oxygen-based evaluation (continuous line) comprises a marked change in contrast to the amplitude-based evaluation. The signals of the two curves begin to divert after about second 225. The oxygen-based curve drops noticeably after about second 310. The behavior in the range between 200 and 300 seconds is a clear indication of hypoxia. The under-supply of oxygen is shown by the rise in the curve. The marked drop in the curve after about second 310 shows a rapid improvement in the oxygen supply.

The pulse signal of these two curves can, moreover, be illustrated with a finer resolution, as is shown for example in FIG. 7, compared with one another, and thus mutually verified, since both signals must show pulse curves that match one another over wide ranges. Errors in the individual measured signals can thus be detected.

FIG. 7 shows an extract from a pleth curve with a higher resolution, and over a shorter period of time. The pulse can be seen clearly. The lower overlaid frequency is the respiration frequency. The correlation between the amplitude-based (dotted) and the oxygen-based (continuous) signals can be clearly seen.

A frequency analysis of the oxygen-based signal, i.e. the one illustrated by a continuous line in FIG. 7, is shown in FIG. 8. The respiration frequency is at about 0.2 Hz, the pulse frequency at about 2.2 Hz. It is generally true that the respiration frequency of adults is significantly lower than the pulse frequency. The pulse frequency is, moreover, recognizable because higher harmonic frequencies can be seen. The existence of such higher frequencies can be used for verification of the pulse frequency.

FIG. 9 shows an exemplary embodiment of a device according to the invention, as described for example in WO2011/161102, for the measurement of blood sugar. The device 1 comprises a housing in which the various optical and electronic components are arranged. The measurement is taken at a finger. The finger is placed into the measuring area 3 or brought up to the measuring area 3. A broadband LED 20 is provided as a source of light, and typically beams light in the spectral range from 400 to 850 nm into the measuring area 3. The housing 16 comprises an opening to let the light out. The opening can be provided with a cover 19 that is transparent to the emerging light. The light that is reflected from or transmitted through the finger is guided into the housing 16 through a second opening in the housing 16, which is also provided with a cover 19 that is transparent to the light. To guide and to disperse the light, a mirror arrangement 5, an aperture slot 7, and a first imaging lens 8 are provided, bringing the light to a diffraction grating 9. The light is dispersed depending on its wavelength by the diffraction grating 9, and is passed through a second imaging lens 10 to the sensor surface 11 of a light detector, in particular an image sensor 12. The image sensor 12 and the LED 20 are arranged on a common circuit board 18 in the housing 16. The circuit board 18 is, moreover, provided with electronic components for controlling the LED 20 and the image sensor 12. In particular the circuit board 18 comprises a USB controller 36, and a USB connection, not shown in more detail. This USB interface permits, on the one hand, a supply of energy to the device 1. On the other hand it enables data exchange with an external computer or display device.

FIG. 10 now shows the schematic structure and the mode of operation of the arrangement of FIG. 9. The device 1 comprises one or more light sources 2 (only one is shown here), which generate measurement light. The light source 2 here serves to illuminate a measuring area 3 to be investigated, typically a region of skin and tissue, as an essentially two-dimensional area with a relatively narrow extent, perpendicularly to its surface. The linear measuring area 3 is thus, in the various forms of embodiment, illuminated by the lighting equipment respectively in either a reflective or transmissive manner, and outputs analysis light 4 according to its transmission or reflection properties. The analysis light 4 is coupled by a diverting mirror 5 into a spectrometer unit for dispersing the light, wherein a spectrometer unit of this sort comprises at least one aperture 7, a first imaging lens 8 and a diffraction grating 9. To determine the oxygen saturation in the blood and other blood values, the analysis light 4 here lies in the range of wavelengths between 400 nm and 850 nm, and comprises a spectral distribution corresponding to the substance composition. The analysis light thus contains spectra in the wavelength range that is relevant to identifying the quantitative substance composition in the measurement area 3, i.e. typically the substance composition of the arterial blood and tissue.

The analysis light 4 reaches an aperture 7 by way of a diverting mirror 5 and a third imaging lens 6. The third imaging lens 6 acts as the inlet objective lens for the spectrometer unit. The aperture 7 has an elongated form, preferably that of a gap or slit, e.g. with a width of typically between 10 μm and 30 μm, and extends horizontally or in the z-direction (perpendicular to the plane of the drawing in FIG. 10). Further optical elements such as filters or additional mirrors may be included in the path of the radiation in order to filter or guide the light.

The strip of the image of the measurement area 3 allowed through by the aperture 7 is projected as light through a first imaging lens 8 onto a diffraction grating 9. For blood value measurements in the context of monitoring, the grating is typically a transmissive “volume phase holographic” grating, with a blaze 35, wavelength in the range from 500 nm to 800 nm and about 300 l/mm to 600 l/mm. The grating 9 is constructed and arranged such that the analysis light 4 is dispersed according to its wavelength, perpendicular to the direction of the slot of the aperture 7, i.e. in the transverse or Y-direction; modified forms of embodiment are accordingly possible here. The diffracted light is projected through a second imaging lens 10 as a diffraction image onto a sensor surface 11 of an image sensor 12. A diffraction image of the aperture 7 or of its slot is thus projected onto the sensor surface 11, with the longitudinal extension of the slot (the z-direction) in one direction and the wavelength-dispersed diffraction image in the other direction. The image sensor for blood value measurements in the monitoring context is typically a CMOS camera sensor of type Aptina MT9m032 (1.6 Mpixels) or MT9P031 (5 Mpixels).

This type of image formation permits the integration through a simple readout. Each line or slot can record the value of a specific wavelength. The integration is completed simply by adding the individual optical values. It is also for example possible to read out and add only the values from preferred areas of the sensor 12, for example those with particular wavelengths. This optical value can, for example, be digitized directly at the sensor 12. Electrical interference can thus to a large extent be excluded or at least minimized. The value calculated in this way can be passed to a processor for conditioning or further processing.

The value can be evaluated in the processor or in further means of evaluation, wherein the processor can be in a suitably programmed computer. It is also possible for the means of evaluation to be part of the device 1, and to be located entirely in it. An external arrangement is conceivable, this arrangement can, for example, have output means such as, for example, a screen or an acoustic indicator.

1-16. (canceled)

17. A method for monitoring the vital parameters of a living being, comprising the following steps:
beaming in broadband light into the tissue of the living being;
recording a spectrum of light radiated back out of the tissue, wherein the light is either reflected or transmitted in the tissue;
carrying out an analysis of the recorded spectrum,
wherein the evaluation comprises the following steps:
determining at least a first parameter on a basis of a first spectral range within the spectrum;
determining at least a second parameter on a basis of at least a second spectral range (I3) which is larger than the first spectral range and which at least partly contains the first spectral range;
comparing the first parameter with the second parameter; and
determining at least one vital parameter on a basis of the comparison.

18. The method according to claim 17, wherein the first spectral range corresponds substantially to a range between at least two neighboring isosbestic points, wherein the absorption coefficient HbO2 is preferably larger than the absorption coefficient HHb in the first spectral range.

19. The method according to claim 18, wherein the first spectral range is composed of at least two partial ranges (I1, I2), wherein an absorption coefficient HbO2 is preferably larger than an absorption coefficient HHb in each partial range (I1, I2).

20. The method according to claim 17, wherein the comparison of the first parameter with the second parameter corresponds to formation of a quotient of the first and the second parameters.

21. The method according to claim 17, wherein the second spectral range (I3) comprises a range of wavelengths at least twice as large as the first spectral range.

22. The method according to claim 17, wherein the second spectral range (I3) comprises a range of wavelengths which comprise a plurality of isosbestic points.

23. The method according to claim 17, wherein the first and the second parameters are determined by an integral of the absorption over the first or the second range (I3).

24. The method according to claim 17, wherein an oxygen signal is generated which represents the evaluation in time-resolved form, preferably with a frequency of 50-150 Hz.

25. The method according to claim 24, wherein the pulse rate is determined from the oxygen signal.

26. The method according to claim 17, wherein a result of the comparison of the first parameter with the second parameter is compared with an amplitude-based signal.

27. The method according to claim 26, wherein said amplitude-based signal is based on the absorption of beamed-in light of a particular wavelength or of a particular range of wavelengths, or with the signal of a pulse oximeter.

28. The method according to claim 17, the broadband light being a pulsed light.

29. The method according to claim 17, wherein the light comprises wavelengths in a spectral range from 400 nm to 850 nm.

30. The method according to claims 17 to 29 for monitoring the pulse rate and/or oxygen saturation.

31. A device (1) for monitoring vital parameters of a living being, comprising at least one light source (2) for beaming light into a tissue, a receiver for light radiation from the tissue, wherein the receiver comprises at least means for dispersing the light, as well as a light detector, so that a light spectrum of the light returned from the tissue can be detected, the device comprises means of evaluation that is designed to determine at least one first parameter on a basis of a first spectral range within the spectrum to be determined, as well as at least one second parameter on a basis of at least one second spectral range (I3), the second spectral range (I3) is larger than the first spectral range, and the means of evaluation is designed to carry out a comparison of the first parameter with the second parameter to determine a vital parameter on a basis of the comparison.

32. The device (1) according to claim 31, wherein said light source is a broadband light source (2) with a wavelength in the range from 400-850 nm.

33. The device (1) according to claim 31, wherein the means of evaluation comprises means for integrating at least the first and the second spectral ranges (I3).

34. The device (1) according to claim 31, wherein the means of evaluation comprises means for forming a quotient from the first and the second parameters.

35. The device (1) according to claim 31, wherein the means of evaluation comprises means for generating an oxygen signal so that the evaluation can be represented in a time-resolved manner.

36. The device (1) according to claim 31, wherein the device (1) comprises a means for fastening the device to one of a finger, an earlobe or to a skin surface.

37. The device (1) according to claim 31, wherein the light detector comprises a 2-dimensional sensor array (12).

38. The device (1) according to claim 37, the light detector is a CMOS sensor.

39. The device (1) according to one of claims 31 to 38 for monitoring the pulse.

40. A device (1) for monitoring vital parameters of a living being, comprising at least one light source (2) for beaming light into a tissue, a receiver for light radiation from the tissue, wherein the receiver comprises at least means for dispersing the light, as well as a light detector, so that a light spectrum of the light returned from the tissue can be detected, the device comprises means of evaluation that is designed to determine at least one first parameter on a basis of a first spectral range within the spectrum to be determined, as well as at least one second parameter on a basis of at least one second spectral range (I3), the second spectral range (I3) is larger than the first spectral range, and the means of evaluation is designed to carry out a comparison of the first parameter with the second parameter to determine a vital parameter on a basis of the comparison, and the carrying out a method as claimed in one of claims 17 to 30.
